(19) United States
(12) Patent Application Publication
(10) Pub. No.: US 2005/0287670 A1
(43) Pub. Date: Dec. 29, 2005

(54) Cell culturing systems, methods and apparatus

(76) Inventors: Eric A. Gulliver, Camarillo, CA (US); Alan N. McGrevy, Oxnard, CA (US); Michael R. Kozlowski, Poway, CA (US); Jerry P. Seymour, Camarillo, CA (US); Philip L. Lichtenberger, Thousand Oaks, CA (US)

Correspondence Address: GREENBERG TRAURIG LLP, 2450 COLORADO AVENUE, SUITE 400E, SANTA MONICA, CA, 90404, US

(21) Appl. No.: 11/088,638

(22) Filed: Mar. 23, 2005

Related U.S. Application Data
(60) Provisional application No. 60/584,761, filed on Jun. 29, 2004.

(51) Int. Cl. C12N 1/21; C12N 5/06; C12N 15/85

(52) U.S. Cl. 435/455; 435/252.3; 435/325; 435/289.1

(57) ABSTRACT

The present disclosure provides cell-culturing methods, apparatus and systems wherein cells in cell cultures are subjected to novel shear forces, which provide improved and efficient target product production. The shear force is provided by a reactor apparatus connected to a cell culture apparatus by a pump for transporting said cell culture from said cell culture apparatus via a first conduit operably connecting said cell culture apparatus to an inlet of a reactor apparatus. The reactor apparatus includes a chamber wherein the cell culture is received and submitted to a shear force as it passes therethrough. The chamber can include a plurality of filters to retain the cells in the cell culture while collecting cell culture media. A stator and a rotor of the reactor apparatus can define the chamber.

### RELATED APPLICATIONS

This application is related to U.S. Provisional Application No. 60/584,761, filed Jun. 29, 2004, the contents of which are incorporated herein by reference in their entirety.

### BACKGROUND

1. Field

An apparatus for cell culturing and associated methods of use is disclosed. More particularly, cell culturing methods, systems and apparatus are disclosed which increase cell culturing efficiency by, for example, improving desired compound production, such as a protein or other desired material, by cells in a cell culture.

2. General Background

Various methods for culturing cell lines are well known in the art. Exemplary methods include solid substrate cell culturing, for example. Such methods utilize cells in a culture that are in contact with and attached to a substrate, over which medium/media, containing nutrients, is passed in order to sustain cells of the culture. Such methods may utilize inert or nutrient containing substrate or substrates that posses particular surface profiles that may promote adhesion/attachment. Media flow over the cells provides cell waste removal and exposure of cells to fresh nutrients and water to “feed” and sustain the cells. Another exemplary cell culturing method is commonly referred to as a suspension culture. In a suspension culture, cells are not attached to a substrate, but instead are maintained in a nutrient-containing fluid suspension/broth, as known in the art.

Various suspension methods have been utilized in the past for the production and maintenance of particular cell lines, such as bacterial cells, neuronal cells, stem cells and mammalian cells, for example. In particular, typical suspension cell culturing methods utilize shakers into or onto which containers, such as beakers or more typically Erlenmeyer flasks containing a cell culturing medium and cells to be cultured, are placed and then shaken. Particular models of shakers can provide incubation of the containers at particular desired temperatures and at particular/variable speeds, thereby providing a range of shaking vigorousness.

Cell culturing can also be accomplished by the use of bioreactors. Such bioreactors typically include a vessel or container in which cells and the cell culturing medium are placed, and further include paddles and/or mixing elements, such as blades, fluid jets, air flow, or fluid flow, that move and circulate the medium and cells contained therein. Exemplary bioreactors include shake flasks, roller bottles, airlift reactors, stirred tank reactors, airlift external loop reactors, tubular loop reactors, surface culture reactors, plunging jet loop reactors, liquid jet reactors, bubble column reactors, packed bed reactors, and membrane reactors (e.g. hollow fiber), for example.

These procedures are typically utilized in order to allow cells in the containers to multiply and generally to provide production of a material/compound of interest, such as a cloned protein produced by a transgene that has been introduced in cells of the culture. Such materials/compounds may be produced and reside within the cells of the culture or be subjected to cellular secretion from the cells and into the surrounding medium, from which such materials/compounds are collected, for example.

Typical techniques utilized to improve cell culture production of a material or compound of interest entail cell-altering techniques such as genetic manipulations or drug treatments, which may modify a material or compound of interest, such as an expressed protein or proteins, for example, in undesirable ways.

Two technologies utilized for increasing protein expression, by placing cells in a non-dividing state, can be characterized as antiproliferative and senescence technologies. Antiproliferative technologies include G1 arrest produced by DNA synthesis inhibitors (e.g. thymidine, hydroxyurea, TGF beta) or genotoxic agents (e.g. adiamycin), G1 arrest produced by conditional mutations (e.g. heat sensitive) and cytostatis produced by tumor suppressor genes (e.g. p27, or p53). Senescent technologies include, for example, premature expression of senescence inducing genes (e.g. p16, p21) and engineered mechanisms for inducing telomere shortening.

Some of these approaches require modifying genomic material of cells of a cell culture. Others approaches require treating the cells with chemicals that have extreme effects on cellular metabolism. Many of these treatments lead to eventual apoptosis. Genetic modifications and chemical treatments have been shown to alter the expression of many proteins in the cell through effects on transcription, translation, or post-translational modification. In addition, pre-apoptotic cells have been demonstrated in many cases to express altered forms of proteins. Perturbations in protein expression can be a problem when trying to express a transfected protein with high fidelity. Apoptosis is also a problem when trying to maintain a cell culture for a sufficient time for use in an industrial protein expression setting.

### SUMMARY

In particular embodiments, a cell culturing system is provided that includes an apparatus having a spinning tube-in-tube arrangement in which a cell culture, including media and cells, is passed through a chamber, such as an annular gap/processing passage provided by and in one aspect of the present disclosure, in order to expose cells in the cell culture to a particular shear force and/or combination of shear forces. In particular embodiments, the inner tube can be provided as a tube having a hollow inner portion or portions or the inner tube can be solid, depending upon a particular desired configuration/use, in accordance with the teachings provided herein.

In some embodiments, the cell culture is passed though a chamber or an annular gap/processing passage that is provided by an inner wall of a stator and an outer surface of a rotor disposed within the stator. The outer rotor wall faces the inner wall of the stator. In particular embodiments, the rotor is mounted concentrically with the stator in order to provide for an annular gap/processing passage that is substantially uniform in dimension throughout, that is from an inlet point to an outlet point. In other embodiments, the rotor is mounted eccentrically with the stator such that the chamber or the annular gap/processing passage changes in dimension (e.g. gap space) from one side or point of the annular gap/processing passage to another point or side of the annular gap/processing passage.

In particular embodiments, maintenance of temperature control of cell culture medium, having cells contained therein, as the medium is introduced and passes though the chamber or the annular gap/processing passage of the apparatus of the present disclosure, is provided by precise heat transfer and control via a temperature transfer fluid, such a heat transfer fluid, that flows and is in contact with an outer wall of the stator and an inner wall of an outer shell/jacket portion of a reactor apparatus disclosed herein. In particular embodiments, the outer shell/jacket includes conduits that provide a counter-current flow of the temperature control fluid around the outer wall of the stator such that the temperature transfer fluid is introduced at approximately the opposite ends of a portion of the apparatus and along the annular gap/processing passage portion of the apparatus. The temperature control fluid flows counter-currently along an axis of the apparatus and in channels that circumnavigate the apparatus and along the length of the annular gap/processing passage, the channels having outlet portions opposite the end of the apparatus into which the temperature transfer fluid was first introduced into the outer shell/jacket portion of the reactor apparatus. In particular embodiments, additional jackets can be provided proximate to and around the conduits circumnavigating the annular gap/processing passage, in order to provide insulation, for example.

Methods of the present disclosure provide for increased efficiency of desired material/compound production by cells in the cell culture. An exemplary desired material/compound can be, but is not limited to, a protein, a peptide, a primary metabolite, a secondary metabolite, a small molecule, etc. or any combination thereof. The teachings of the present disclosure expose cells in cell culture to shear forces that provide disaggregating forces that act on cells in culture media. The teachings also provide for an improved cellular microenvironment. The present disclosure provides beneficial cellular stresses, which result in improved material/compound production by cells of the cell culture.

The present disclosure also provides an apparatus and methods for reducing rates of cell division in the cell culture and hence increasing the production of at least one desired material/compound by these cells. In particular embodiments, desired material/compounds are encoded by at least one transgene introduced into the cells of the cell culture.

The present disclosure also provides an apparatus and methods for maintaining rates of cell division of cells in the cell culture at or near control and/or wild type levels. In one aspect, cell populations can be exposed to shear forces in accordance with the present disclosure, increase in production of at least one desired material/compound by these cells.

A cell culturing system in accordance with the present disclosure also provides a system that can be retrofitted to existing cell culture platforms such as bioreactors for example or other cell culturing platform and can bring additional functionality to such a host system.

### BRIEF DESCRIPTION OF THE DRAWINGS

The foregoing aspects and embodiments of this disclosure will become more readily appreciated as the same becomes better understood by reference to the following detailed description, when taken in conjunction with the accompanying drawings, wherein:

FIG. 1 illustrates exemplary cell culturing system including a schematic of one embodiment of an illustrative reactor apparatus in accordance with the present disclosure;

FIG. 2A illustrates the right side of an illustrative reactor apparatus;

FIG. 2B illustrates the left side of the illustrative reactor apparatus;

FIG. 3 illustrates a partial internal view of an illustrative reactor apparatus;

FIG. 4 illustrates an exploded view of components of an illustrative reactor apparatus;

FIG. 5A illustrates a cross-sectional view of the illustrative reactor apparatus along line 5A-5A of FIG. 3;

FIG. 5B illustrates a close-up cross sectional portion of the reactor apparatus shown in FIG. 5A;

FIG. 6A illustrates an illustrative cross section view of a shear treatment zone and inlet at a portion of an annular gap/processing passage in an illustrative reactor apparatus;

FIG. 6B illustrates another cross section view of a shear treatment zone and outlet at another portion of an annular gap/processing passage in an illustrative reactor apparatus;

FIG. 7 illustrates a close-up cross-section view of an exemplary configuration of a temperature control exchanger that conducts a fluid through conduits for controlling and/or maintaining a desired temperature;

FIG. 8A illustrates a left side view of conduits providing a flow path for cooling/heating fluid in a body portion of an illustrative reactor apparatus;

FIG. 8B illustrates a right side view of conduits providing a flow path of cooling/heating fluid in a portion of the body of an illustrative reactor apparatus;

FIG. 9 illustrates a diagram depicting increased protein production by a treated cell culture when compared to controls, in accordance with principles of the present disclosure;

FIG. 10 illustrates a diagram depicting increases in cell number under cell culturing conditions in accordance with the present disclosure compared to control cell-culturing conditions; and

FIG. 11 illustrates a diagram exemplifying increased desired material/compound production by cells in a cell culture, here exemplified by protein production, in accordance with the teachings of the present disclosure.

### DETAILED DESCRIPTION

Particular embodiments are described below in considerable detail for the purpose of illustrating various teachings, principles and methods of operation. However, various modifications may be made and the scope of the disclosure is not limited to exemplary embodiments described herein.

In particular embodiments, various conventional culturing systems are operably connected to a reactor apparatus having a chamber or an annular processing gap/processing passage though which cell culture medium is passed. U.S. Pat. Nos. 5,279,463, 5,538,191, 6,471,392 and 6,742,774 disclose various apparatus that can be modified and utilized in accordance with various aspects of the teachings of the present disclosure. In the apparatus and system disclosed herein, temperature control of a cell culture is precisely controlled as the cell culture passes through the annular gap/processing passage of the reactor apparatus. In another aspect, the apparatus disclosed herein also provides a new perfusion method.

Exemplary perfusion includes removal of medium from a bioreactor and its replacement while retaining the cells in the bioreactor. Thus, perfusion fundamentally relies on separating the cells from the medium in which they are maintained and into which they may secrete at least one desired material/compound or compounds that they produce, such as, but not limited to, a protein, for example. Perfusion can serve many important functions in protein production, including allowing harvesting of a desired protein produced by the cells, replenishment of cell culture medium, and removal of harmful byproducts and/or cellular waste. Separation of the cells and medium is difficult to achieve on a production scale because cells are small, sticky, delicate, and very similar to the medium in density.

Three major methods of separation are settling out the cells, centrifugation, and filtration. Attempting to settle or spin out the cells is slow and can cause cell death either due to the centrifugal force or to insufficient media bathing of the cells. Employing prior art methods to filter the medium (e.g. with membranes) leads to plugging of the membranes by the cells, which then die.

In accordance with one aspect of the present disclosure, a perfusion method disclosed herein combines features of centrifugation and filtration approaches. The medium passes out of a circulating system through at least one opening (not shown in the drawings) in rotor 129 that includes at least a portion thereof having a membrane, such as a protein permeable filter, or other type of cell-retaining filter. However, the system and process described herein is not a reverse osmosis process, and reverse osmosis filters are not appropriate. Exemplary useful membranes include those having pore sizes of 0.0032 μm to 10 μm with common ranges between 0.2 μm to 4.5 μm, and may be a cartridge type or a flat membrane type, such as those sold by Critical Filtration, Inc., High Purity Solutions, Qtec, Sartorius and Microfiltration Media. Useful filters could be constructed out of polypropylene, polyvinyldiene fluoride, polyethersulfone, cellulose, or a similar material. These are standard filters used in sterilization, and can be obtained from a number of suppliers including Millipore, Pall Corporation, and Sartorius. Cells are prevented from plugging the filter by the mild centrifugal force acting in the annular gap/processing passage 152, which moves them away from the rotor and towards the stator. The cell-retaining, medium transporting membrane is disposed over the openings in the rotor 129, which can be circular, square, elongated, or any useful shape. Rotor 129 in this particular embodiment may be hollow. Flow of media out of an annular gap/processing passage 152 is accomplished via a conduit to a collection vessel. Additional media can be added to the processing passage via addition of media to vessels in cell culture apparatus 40 that contain the cell culture in appropriate retaining vessels (e.g. tanks, flasks, beakers, etc.) As the perfusion takes place through the membrane attached to and around the outside diameter of the rotor, it is passed into a small chamber and transferred through a series of stationary tubes inside the hollow rotor. The tubes pass rearward through the center of the rotor. The fluid or media perfused is transferred from the rear of the rotational rotor shaft by means of a rotary coupling such as, but not limited to, Rotary Systems, Inc. part #10010 rotary coupling for fluid transfer. The structure may include one or more passages. DSTI is a supplier of custom rotary couplings meeting FDA standards. A small vacuum or a peristaltic pump may be used to assist passage of fluid through the system.

In one aspect, the reactor apparatus of the present disclosure does not require genetic manipulations or drug treatments to achieve stasis and/or enhanced protein production by cells of a cell culture. Instead, the reactor apparatus disclosed herein employs an applied force that activates a natural pathway within cells of the cell culture. Because a natural pathway is used, the system and cell treatments of the present disclosure are less likely to broadly alter the expression of proteins or other desired materials by cells in a cell culture, including those produced by transfected genes. In addition, cell viability is not lowered by the present treatment. Thus, the cells can be maintained in culture for extended periods of time necessary for industrial production.

It is noted that in typical cell culture apparatus, such apparatus will induce some shear force and some turbulent mixing force on cells. In a shake flask system, there will be a mild shear force associated with a velocity gradient in a boundary layer at the edge of the flask, and a turbulent force associated with the orbital motion. In a stirred tank bioreactor, cells will experience shear force as they pass near the mixer blades and mixing forces elsewhere in the vessel. In all standard bioreactors, these forces are considered “mild” since essentially no cell death is attributed to them. In fact, the goal of a good bioreactor is to minimize cell stress.

However, cells treated in accordance with one aspect of the present disclosure (e.g. passing through the annular gap/processing passage of the reactor apparatus while the rotor is spinning) have a qualitatively different experience of force, which results in a different kind of cell stress. Variables of this force for influencing cells stasis and/or protein production include degree, duration and uniformity of the applied force.

For one, cells passing through the reactor apparatus are exposed to shear forces that are produced by boundary layers at the wall of the stator and the wall of the rotor that are either contacting or overlapping within the chamber or annular gap/processing passage through which the cells in the cell culture medium pass. The cells are exposed to a single uniform shear field unlike that seen in shaker flasks or stirred containers where the shear force varies with both position and time. When the annular gap is in the size range described for this invention, flow within the gap is laminar, with no Taylor vortices. Thus, the shear force that the cells experience is remarkably uniform. For example, cells do not pass through regions of high shear force in the laminar zone, through regions of a different shear force in the Taylor currents, to regions of low shear force in the eddies of the Taylor vortices. Moreover, since all cells spend the same amount of time in the gap, the cumulative shear force that they experience is likewise uniform. In an environment in which there is a plurality of shear forces such as a stirred bioreactor, different cells might be exposed to different amounts of shear for different periods of time, depending on their location in, and motion through, the environment. The present invention, by allowing a precise amount of shear stress to be universally applied, permits a non-dividing and/or more productive state to be induced in the culture without significant loss of viability. In an environment with a plurality of shear forces, inducing the same “average” stress level as the current invention would require cells to spend some time in higher shear zones, which might decrease viability, to balance their time in lower shear zones that might be ineffective in altering the state of the cells.

As such and in one embodiment, reactor apparatus 100 of the present disclosure can increase protein production by fifty percent or more using the uniquely provided mechanical stimulation experienced by cells of the cell culture as they pass through annular gap/processing passage 152. The ability of this treatment to induce cell stasis under certain conditions may have additional benefits. First, there is a growing body of evidence indicating that non-dividing cells are more efficient at producing desired materials, such as cloned proteins. Second, use of non-dividing cells allows the cell culture to be set up with higher cell density, for better early production, and maintained indefinitely without having to remove cells. That is, higher early production can be achieved without having to wait and allow for sufficient time to pass for a relatively low number of cells to divide and multiply to a high/critical number of cells in order to provide significant production of a desired material.

The forces generated by the turning of rotor 128 within stator 129 of reactor apparatus of the present disclosure provide and maintains dis-aggregation of cells in cell culture. As mentioned above, it has been shown that cells growing in clumps tend to be less productive because of poor perfusion of cells in the center of the aggregates. The apparatus, systems and methods disclosed herein provide an improved cellular microenvironment. The outside of cell membranes can trap spent media, products, wastes, etc. which conventional mixing or airlift may not be vigorous enough to release. Furthermore, the apparatus, systems and methods disclosed herein provide particular and novel cell stress which increases cellular production of at least one target element/compound by cells in a cell culture, here exemplified in one embodiment by a transfected gene encoding a protein.

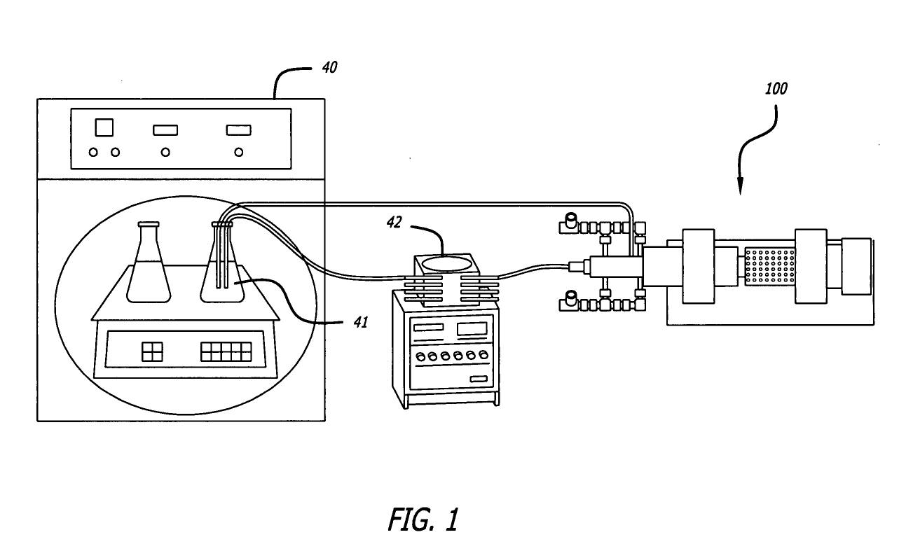

FIG. 1 illustrates an exemplary cell culturing system, which includes an illustrative reactor apparatus 100 in accordance with one aspect of the present disclosure. The reactor apparatus 100 is in communication with a shaker flask 41 via a provided conduit, such as standard tubing utilized in biological laboratories, as known in the art. Shaker flask 41 sits on a shaker platform located inside a cell culture apparatus 40. As shown in FIG. 1 and in this embodiment, the system is a closed (e.g. circulating) system, where a cell culture is circulated between a cell culture apparatus 40 and the reactor apparatus 100. In one embodiment, reactor apparatus 100 is in operable communication with a pump 42, such as a peristaltic pump for example, and cell culture apparatus 40. In one embodiment, the direction of the control flow can be established by transferring at least a portion of the cell culture from a flask 41 to pump 42, then to reactor apparatus 100, and back to a retaining vessel, such as a flask 41 or to another flask (i.e. a flask or vessel other than the vessel from which the at least portion of the cell culture originates). In another exemplary embodiment, in configurations where cells do not circulate (e.g. are not conducted through a closed circulating circuit), the culture medium can be maintained in a vessel (not shown), passed through the reactor apparatus 100, and then into a receptive vessel, such as flask 41 and/or another vessel, where the cell culture is maintained or subjected to further desired treatments. Other configurations are contemplated. The pump 42 permits the circulation of the cell culture from the illustrative cell culture apparatus 40 having a shake flask system (for example in FIG. 1) to the reactor apparatus 100. Of course, the flow of cell culture medium in a closed circulating system, as illustratively shown in FIG. 1, can run in either direction (i.e. clock-wise or counter-clockwise). As would be known to persons skilled in the art, in order to accomplish flow in the opposite direction, the ports and other components would need to be reconfigured to permit flow and processing in the opposite direction.

The pump 42 can also be a gear pump, syringe pump, multi-piston pump, pressure pump, etc. In this embodiment, cell culture apparatus 40 can be a conventional “shake flask” incubated system, but other cell culture apparatus that are contemplated include, but are not limited to, roller bottles, airlift reactors, stirred tank reactors, and airlift external loop reactors, tubular loop reactors, surface culture reactors, plunging jet loop reactors, liquid jet reactors, bubble column reactors, packed bed reactors, and membrane reactors (e.g. hollow fiber). With attached cell growth systems, such as surface culture, packed bed, and membrane reactors, the cells would be circulated/passed through the reactor apparatus 100 at least once before establishing fixed growth within a reactor for attached cell growth.

In the exemplary system shown in FIG. 1, cells in the cell culture are exposed to a plurality of shear forces. A first shear force on the cells of the culture is provided by the boundary layer at the edge of the flask and turbulent force associated with the orbital motion of the shake flask system of cell culture apparatus 40. As the cells of the culture are pumped out of the cell culture apparatus 40 and into the reactor apparatus 100, they are exposed to another set of shear forces provided by the boundary layers of the rotor 128 and stator 129 walls of the reactor apparatus 100. The profile of the force (a combination of degree and duration) that cells experience in the reactor apparatus 100 is much greater than that induced by a conventional bioreactor. Data indicates that the reactor apparatus 100 can produce massive cell death if the rate of rotation and, thereby, the shear force that is exerted on the cells, is moderately increased over a maximum tolerance level of a particular cell culture.

As known in the art, there are various aspects to fluid flow in an annulus such as the annular gap/processing passage 152 disclosed herein, such as Reynolds Numbers, Taylor Numbers, shear rates and shear stress. The literature lists several versions of both Reynolds number (Re) and Taylor number (Ta). These dimensionless numbers are ultimately ratios of the momentum in a fluid flow and the viscous forces in the fluid. When viscous forces dominate, flows tend to be laminar and Re and Ta are low, but when momentum dominates the flow, the flow tends toward turbulence and Re and Ta are high. The different versions of Re and Ta result from adapting the equations to different flow configurations. Thus, there are Reynolds numbers for pipe flow, flow in a slot, axial annular flow and tangential annular flow. In the case of a stator 129 and rotor 128, Taylor numbers are specific to annular flow with an inner cylinder (e.g. rotor 128) rotating, but the definitions appear to vary especially when it comes to the critical Taylor numbers used to define the transitions between laminar and turbulent flow.

Taylor rings (vortices) have not been observed in a rotor/stator device/process as described herein. In addition, the calculations that follow suggest that the experimentally determined upper bound of the preferred conditions may coincide with the formation of Taylor vortices. The lack of Taylor rings allows us to apply a uniform shear force. If there are Taylor rings, it means there will be both currents and eddies. Each of these will apply a different amount of shear. The laminar flow generating the Taylor vortices will apply a third amount of shear. Cells are very sensitive to shear, so the goal is applying enough shear to achieve the desired effect on division and protein production but not so much that the cells become non-viable. In a non-uniform shear field, this will not be possible since, although the average amount of shear might seem correct, the higher shear areas will induce non-viability, while the lower shear areas may do nothing.

The boundaries between laminar and turbulent flow seem to coincide with the upper boundary of the preferred conditions as outlined below. The discussion of the different versions of Reynolds and Taylor numbers are included as a way to reconcile differences and to show that they tend to coincide with the experimentally determined upper bound of the preferred conditions, and to provide a method for determining operation conditions in different mechanical configurations with different cell cultures and media. Bird, Stewart and Lighffoot (“Transport Phenomena”, R. B. Bird, W. E. Stewart and E. N. Lighffoot, John Wiley & Sons, New York, (1960), pp. 96) define the Reynolds number for tangential flow in an annulus (Re_{BSL}) and the critical Reynolds number for transition from laminar to turbulent flow as:

$$R {e}_{BSL} = ( \frac{Ωκ {R}_{0}^{2} ρ}{μ} ) {• ( \frac{41.3}{{( 1 - κ )}^{3 / 2}} )}_{critical} \qquad ( 0.1 )$$

Where:
Ω=angular velocity of the inner cylinder (radians/s or 1/s),
κ=radius of the inner cylinder divided by radius of the outer cylinder (none),
Rₒ=radius of the outer cylinder (m),
ρ=fluid density (kg/m³) and
μ=fluid viscosity (kg/m s)

Defined also is the Reynolds number for axial flow in an annulus (Re_{z}) (on pp. 54) and note that the transition from laminar to turbulent flow occurs at Reynolds numbers of about 2000. For example and in this case, the Reynolds number is:

$$R {e}_{z} = \frac{2 {R}_{0} ( 1 - κ ) 〈 {υ}_{z} 〉 ρ}{μ} \qquad ( 0.2 )$$

Where:
Rₒ=radius of the outer cylinder (m),
κ=radius of the inner cylinder divided by radius of the outer cylinder (none),
<ν_{z}>=average fluid velocity in axial direction (m/s),
ρ=fluid density (kg/m³) and
μ=fluid viscosity (kg/m s).

In another work, Kataoka (Taylor Vortices and Instabilities in Circular Couelte Flows”, K. Kataoka, pp 236-274, in Encyclopedia of Fluid Mechanics, Vol. 1 Flow Phenomena and Measurement, Ed. N. P. Chereminisinoff, Gulf Publishing Co., Houston, (1986), on p. 238) defines the Reynolds number for tangential flow (Re_{K}) as:

$$R {e}_{K} = \frac{{R}_{i} Ω d}{v} = \frac{{R}_{i} Ω d ρ}{μ} \qquad ( 0.3 )$$

Where:
Rᵢ=inner cylinder radius (m),
Ω=angular velocity of inner cylinder (1/s),
d=annulus gap width (m),
ν=fluid kinematic viscosity [Note: ν=μ/ρ] (m²/s),
μ=fluid viscosity (kg/m s) and
ρ=fluid density (kg/m³)

At the same time Kataoka defines the Taylor number (Ta_{K}) as:

$$T {a}_{K} = \frac{{R}_{i} {Ω}^{2} {d}^{3}}{{v}^{2}} = \frac{{R}_{i} {Ω}^{2} {d}^{3} {ρ}^{2}}{{μ}^{2}} \qquad ( 0.4 )$$

Where,
Rᵢ=inner cylinder radius (m),
Ω=angular velocity of inner cylinder (1/s),
d=annulus gap width (m),
ν=fluid kinematic viscosity [Note: ν=μ/ρ] (m²/s),
μ=fluid viscosity (kg/m s) and
ρ=fluid density (kg/m³)

In this work, Kataoka goes on (p. 243) to define the critical Taylor number (Ta_{c}) as the threshold below which “infinitesimal disturbances are damped owing to the action of viscosity” and above which “some of them are amplified with increasing time”. This is taken to mean that Ta_{c} is the threshold for formation of Taylor rings (a.k.a., Taylor vortices). It is stated therein, that for very narrow gap widths (i.e., d/rᵢ<<1) Ta_{c} approaches 1,708 but that it tends to increase with increasing d/Rᵢ.

In an example, he states that when d/rᵢ=0.33, Ta_{c}=2,453. Kataoka provides two equations for estimating Ta_{c};

$$\begin{matrix}T {a}_{c} = \frac{{π}^{4} ( 1 + \frac{d}{2 {R}_{i}} )}{0.0571 ( 1 - \frac{0.652 d}{{R}_{i}} ) + 0.00056 {( \frac{0.652 d}{{R}_{i}} )}^{- 1}} \\ and , for d ⪡ {R}_{i} ; & ( 0.5 ) \\ T {a}_{c} = 1695 ( 1 + \frac{d}{2 {R}_{i}} ) & ( 0.6 )\end{matrix}$$

where,
d=annulus gap width (m), and Rᵢ=inner cylinder radius (m).

Kataoka further notes that as the rotor rpm increases, the Taylor rings become unstable such that the vortex boundaries are S-shaped or wavy. There is a second critical Taylor number (Ta_{w}) and a second critical Reynolds number (Re_{w}) that corresponds to this instability. Ta_{w} and Re_{w} both depend on the radius ratio of the rotor and stator (η=Rᵢ/Rₒ).

Schlichting (“Boundary-Layer Theory”, 7^{th} ed., H. Schlichting (translated by J. Kestin), McGraw-Hill, Inc., New York, (1955) [Reissued in 1987] on pp. 526-529) gives the following equation for the Taylor Number (Taₛ):

$$T {a}_{S} = \frac{U d}{v} \sqrt{\frac{d}{{R}_{i}}} = \frac{U d ρ}{μ} \sqrt{\frac{d}{{R}_{i}}} \qquad ( 0.7 )$$

Where,
U=the peripheral or surface velocity of the inner cylinder (m/s),
d=the gap width between the two concentrically placed cylinders (m),
ν=fluid kinematic viscosity (m²/s),
Rᵢ=radius of the inner cylinder (m),
ρ=fluid density (kg/m³) and μ=fluid viscosity (kg/m s).

Schlichting gives Ta_{c} as 41.3 and states that for 41.3<Ta<400 flow is laminar with Taylor vortices while flows with Ta>400 are turbulent.

Bird, Stewart and Lightfoot's tangential Reynolds number, Kataoka's Taylor number and Schlichting's Taylor produce widely different values for the same flow conditions but if the results are examined in terms of the transition or critical numbers provided with each equation the results are in close agreement. In other words, if Bird, Stewart and Lightfoot's tangential Reynolds number is larger than the critical value for flow instability, Kataoka's and Schlichting's Taylor numbers will be greater than their respective critical values as well.

A guiding principle of fluid mechanics is the no slip rule. This states that fluid in contact with a surface moves at the same velocity as the surface. This produces a velocity in a fluid bounded by two surfaces when one surfaces moves relative to the other. This gradient is called shear rate and is a useful measure of how intensely a material is sheared. Shear Rate (y) has units of s⁻¹ and is a function of the rotor surface velocity and the rotor-stator gap.

$$γ = \frac{U}{d} \qquad ( 0.8 )$$

Where,
U=the peripheral of surface velocity of the inner cylinder (m/s) and
d=the gap width between the two concentrically placed cylinders.

Shear stress (σ) is a measure of the shearing force applied to a material and has units of kg/m s². Since it reflects the force applied to a fluid, it is more likely to reflect the impact a given set of flow conditions will have on cellular organisms. It is possible to subject a fluid to high shear rates but low shear stress. This is because shear stress is a function of shear rate and viscosity.
σ=μγ (0.9)
Where,
μ=viscosity (kg/m s) and γ=shear rate.

In one embodiment, the reactor apparatus 100 disclosed herein has an outer cylinder (stator 129) radius of 0.794 cm. The annular gap/processing passage 152 (rotor/stator gap) could be varied from about 254 micrometers to 457 micrometers. The inner cylinder (rotor 128) RPM was varied from 250 RPM to 1800 RPM, with a preferred RPM being between 400 RPM and 600 RPM. The viscosity of the cell culture was taken to be similar to whole blood since both systems consist of cells suspended in complex mixtures of nutrients. Literature values for blood viscosity range from as low as 3.2 centipoises to as high as 400 centipoises. This is probably because blood is a shear thinning fluid and its viscosity decreases as the shear rate increases. It is also conceivable that a cell culture viscosity could approach 1 centipoise if there are very few cells and nutrients in the broth. The density of whole blood ranges from 1.043 g/cm³ to 1.066 g/cm³. Exemplary maximum and minimum values of γ, σ, Re_{BSL}, Ta_{K} and Ta_{S} for these conditions are summarized in Table 1.

TABLE 1
Maximum and minimum values of shear rate, shear stress,
Bird, Stewart and Lightfoot (BSL) Reynolds number, Kataoka
Taylor number and Schlichting Taylor Number*.

| | Shear | Shear | Reynolds | Taylor | Taylor |
|---|---|---|---|---|---|
| | Rate | Stress | Number - | Number - | Number - |
| | (γ) | (σ) | BSL | Kataoka | Schlichting |
| | [s⁻¹] | [kg/m s²] | (Re_{BSL}) | (Ta_{K}) | (Ta_{S}) |
| Minimum | 428 | 0.43 | 4 | 0.001 | 0.02 |
| Maximum | 5,702 | 2,280 | 12,254 | 28,863 | 170 |

*For operating conditions using an exemplar stator internal diameter of 1.588 cm and where annular gap/processing passage 152 (rotor/stator gap) ranged from 254-457 microns, rotor RPM ranged from 250-1800 RPM, fluid viscosity ranged from 1-400 centipoises and fluid density ranged from 1.043-1.066 g/cm3.

In one embodiment, as an example, a rotor 128 RPM is between 400 and 600 RPM and the annular gap/processing passage 152 (rotor/stator gap) is between 254 and 381 microns. Other operating parameters are shown below Table 2.

The maximum and minimum values of γ, σ, Re_{BSL}, Ta_{K} and Ta_{S} for these conditions are summarized in Table 2.

TABLE 2
Maximum and minimum values of shear rate, shear stress,
Bird, Stewart and Lightfoot (BSL) Reynolds number, Kataoka
Taylor number and Schlichting Taylor Number*.

| | Shear | Shear | Reynolds | Taylor | Taylor |
|---|---|---|---|---|---|
| | Rate | Stress | Number - | Number - | Number - |
| | (γ) | (σ) | BSL | Kataoka | Schlichting |
| | [s⁻¹] | [kg/m s²] | (Re_{BSL}) | (Ta_{K}) | (Ta_{S}) |
| Minimum | 831 | 0.83 | 6.56 | 0.002 | 0.04 |
| Maximum | 1,901 | 760 | 4,085 | 1,875 | 43 |

*For operating conditions an exemplar stator internal diameter of 1.588 cm and where annular gap/processing passage 152 (rotor/stator gap) ranged from 254-381 microns, rotor RPM ranged from 400-600 RPM, fluid viscosity ranged from 1-400 centipoises and fluid density ranged from 1.043-1.066 g/cm3.

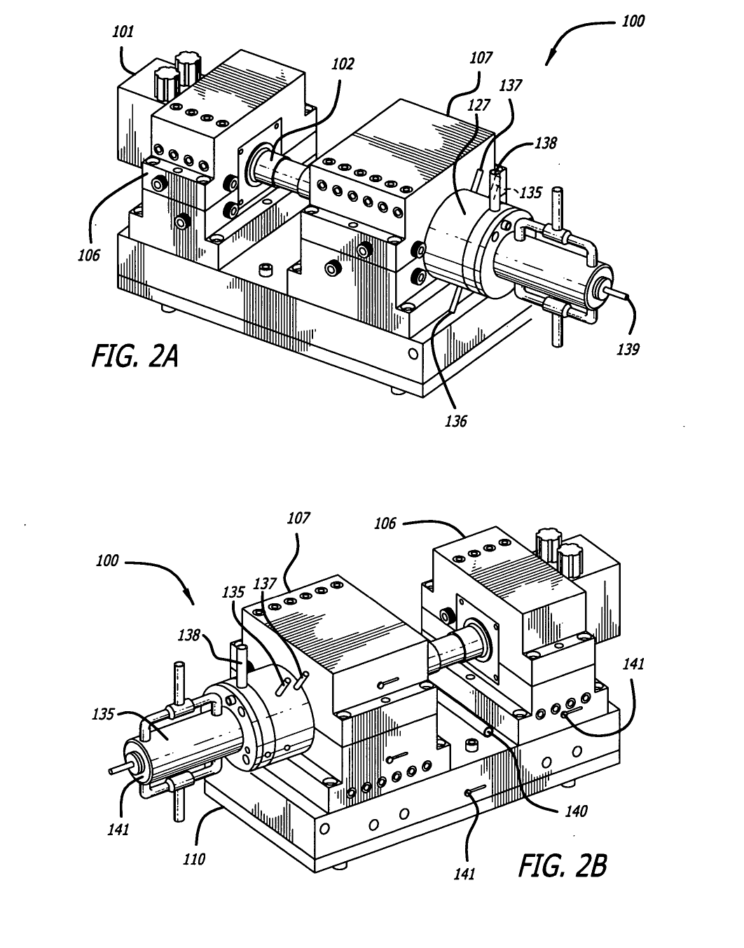

It is worth noting that the maximum values for the Reynolds and Taylor numbers in both Tables 1 and 2 are greater than the critical values for the transition from laminar flow to Taylor rings because of the broad range of viscosity and RPM assumed. As shear increases with higher RPM, the fluid viscosity approaches 1 (one) centipoise and therefore the laminar condition is maintained within this operating range. The various parameters may be adjusted according to the working fluid properties, so long as no Taylor vortices are formed during processing within the annular gap 152. FIGS. 2A and 2B respectively illustrate the right side and the left side of an illustrative reactor apparatus 100. In this embodiment, the reactor apparatus 100 employs a motor 101 that is configured to produce the rotating force on the rotor 128 of the reactor apparatus 100. The reactor apparatus 100 also includes a motor chill block 106 and a bearing chill block 107 that rest on a base plate 110. The motor chill block 106 contains a plurality of conduits that provide cooling to the motor 101 via passage of a temperature control fluid, typically a cooling fluid, therethrough. The motor 101 connects to bearings through a coupling 102. The bearing chill block 107 also contains a plurality of conduits that provides cooling and lubricant to the bearings located inside the motor chill block 107. A front seal block 127 has a cylindrical shape and an opening for bearing oil inlet 137. Shown also are a process outlet 138, from which a cell culture exits after passage through annular gap/processing passage 152 (not shown in this view), a cooling/heating fluid inlet 135, and a cooling/heating fluid outlet 136. Bearing oil is utilized primarily to lubricate the bearings. The cooling/heating fluid used in the bearings allows for temperature regulation of the bearings. In one embodiment, the reactor apparatus 100 has at least one, preferably a plurality of temperature sensors 141 located and disposed at various locations, one such location shown in FIG. 2B, in order to monitor temperature of various portions of apparatus 100 and prevent overheating and malfunction. It would be known to persons skilled in the art to add other temperature sensors in other locations as desired.

The reactor apparatus 100 also includes a primary inlet 139 which is used to introduce the cell culture medium containing cells to be treated into the annular gap/processing passage 152 (not shown here). A process outlet 138 releases the cell culture after passage through the annular gap/processing passage of reactor apparatus 100. In one embodiment, annular gap/processing passage 152 can be about 4 inches long (from process inlet to process outlet), although other lengths are contemplated ranging from 0.5 inches up to about 3 feet. In this one embodiment, a single primary inlet 139 is depicted and provided in-line with stator 129 and rotor 128. In other embodiments, primary process inlet 139 can be provided substantially perpendicular to stator 129 and rotor 128, such that the cell culture is introduced perpendicularly to the axis of stator 129 and rotor 128. In still other embodiments, a plurality of inlets can be provided, configured both in-line and perpendicular to the axis defined by stator 129 and rotor 128, respectively, providing the ability to feed at least a portion of a cell culture into the annular gap/processing passage 152 at two points, for example. Of course, three or more insert points can be provided, if so desired. Furthermore, and in alternative configurations, reactor apparatus 100 can include a single outlet with a single or multiple inlets, or a plurality of outlets and a single inlet can be provided.

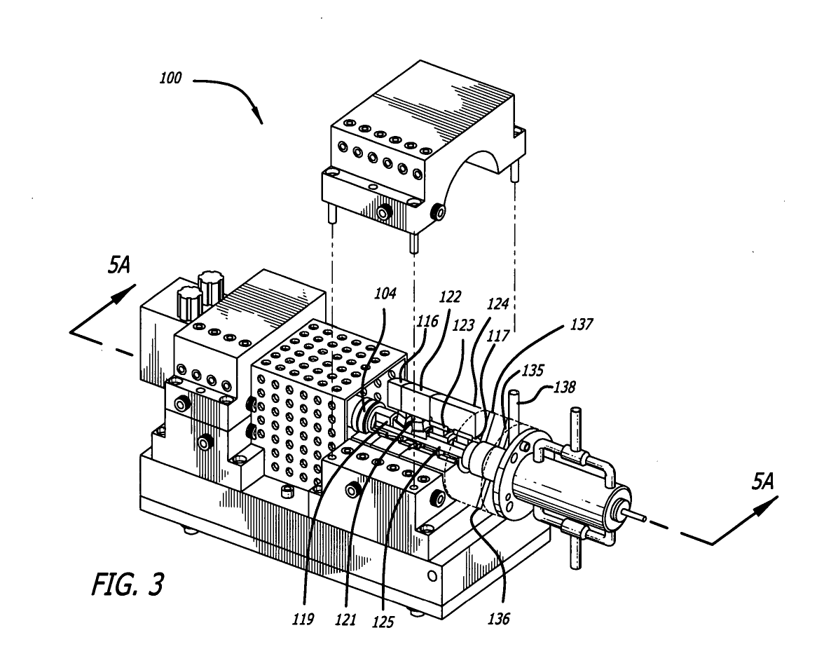

FIG. 3 illustrates a partial internal view of the reactor apparatus 100. In particular, the partial internal view depicts and corresponds to bearings connected to the front seal block 127. The components of a tapered bearing block 122 and of a roller bearing block 124 include numerous bearings, rollers, screws and seals that are coupled and interconnected to transmit the rotational force from motor 101 to the front seal block 127.

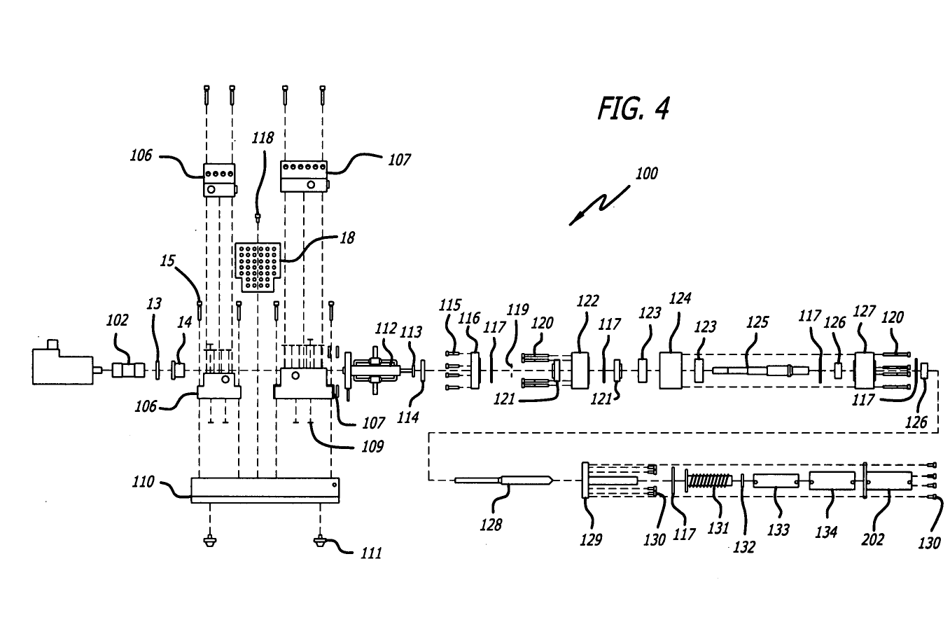

FIG. 4 illustrates an exploded view of some components of the reactor apparatus 100. In this embodiment, the various parts and quantities that comprise a particular illustrative embodiment of the reactor apparatus 100 are identified by the corresponding reference number, quantity and description provided below. The parts are sized as appropriate for the particular apparatus configuration chosen.

| Ref. No. | QTY | DESCRIPTION |
|---|---|---|
| 101 | 1 | Motor |
| 102 | 1 | Coupling |
| 103 | 1 | Jam Nut |
| 104 | 1 | Lock Nut |
| 105 | 16 | Hex Screw |
| 106 | 2 | Motor Chill Block |
| 107 | 2 | Bearing Chill Block |
| 108 | 1 | Protection Shield |
| 109 | 16 | O-Ring |
| 110 | 1 | Base Plates |
| 111 | 4 | Rubber Foot |
| 112 | 1 | Draw Bar |
| 113 | 1 | Washer |
| 114 | 1 | Seal |
| 115 | 1 | Hex Screw |
| 116 | 1 | Rear Seal Plate |
| 117 | 5 | O-Ring |
| 118 | 2 | Hex Screw |
| 119 | 1 | O-Ring |
| 120 | 8 | Hex Screw |
| 121 | 2 | Tapered Bearing |
| 122 | 1 | Tapered Bearing Block |
| 123 | 2 | Roller Bearing |
| 124 | 1 | Roller Bearing Block |
| 125 | 1 | Collet |
| 126 | 2 | Seal |
| 127 | 1 | Front Seal Block |
| 128 | 1 | Rotor |
| 129 | 1 | Stator |
| 130 | 8 | Hex Screw |
| 131 | 1 | Spiral Manifold |
| 132 | 1 | O-Ring |
| 133 | 1 | Stainless Steel Sleeve |
| 134 | 1 | TEFLON ® Insulator |
| 202 | 1 | Outer Shell |

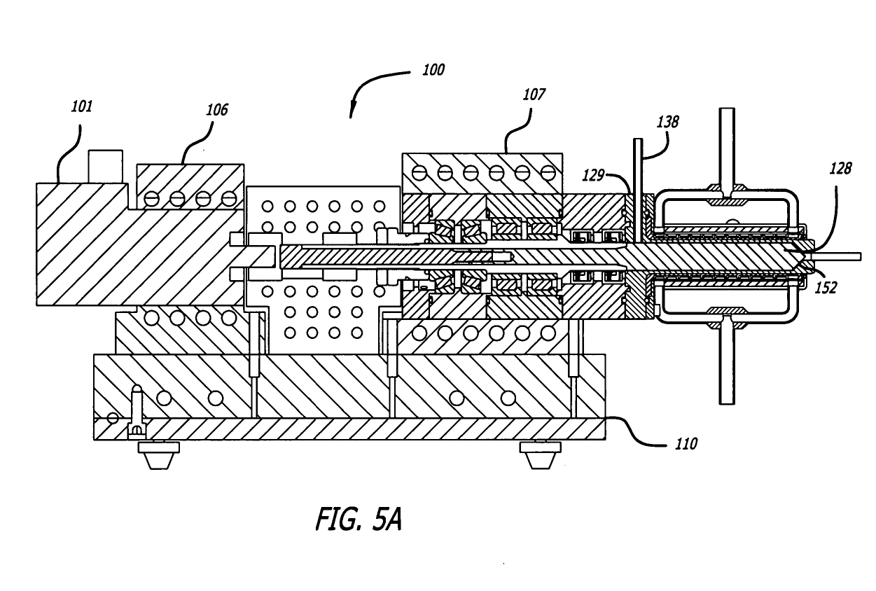

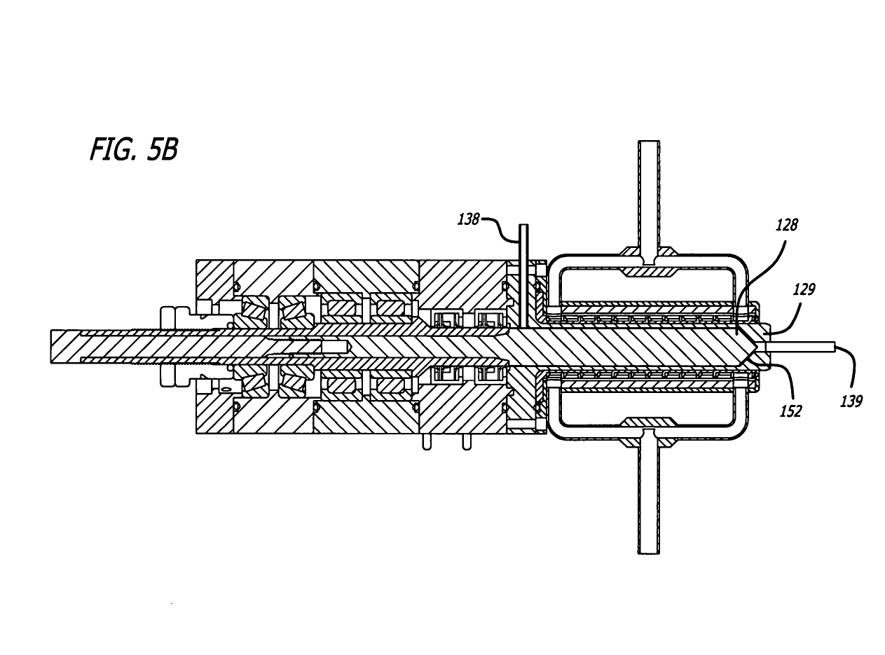

FIG. 5A illustrates a half section view of the reactor apparatus 100 along line 5A of FIG. 3. The half section view illustrates the motor 101, the motor chill block 106, the bearings chill block 107, the base plate 110, etc. Also, the reactor apparatus 100 shows previously listed components assembled together. In particular, a rotor 128 and a stator 129 are shown assembled together to provide annular gap/processing passage 152 of reactor apparatus 100.

FIG. 5B illustrates a close-up of a portion of the half section view of the reactor apparatus 100 shown in FIG. 5A. The external surface of the rotor 128 and the interior surface of the stator 129 define an annular gap/processing passage 152 through which the cell culture medium, containing cells, pass. In one aspect, a cell culture is introduced through primary inlet 139, processed through gap 152 and released through process outlet 138.

The rotor 128 and stator 129 of the reactor apparatus 100 can be made out of “super alloys” or “high performance alloys” (such as Hastelloy C®) or stainless steel 316L. Other metals such as titanium, or any material that can be autoclaved, or decontaminated can also be utilized. Such materials preferably have non-porous surfaces so that effective sterilization of rotor 128 and stator 129 surfaces that come into contact with cell cultures is realized.

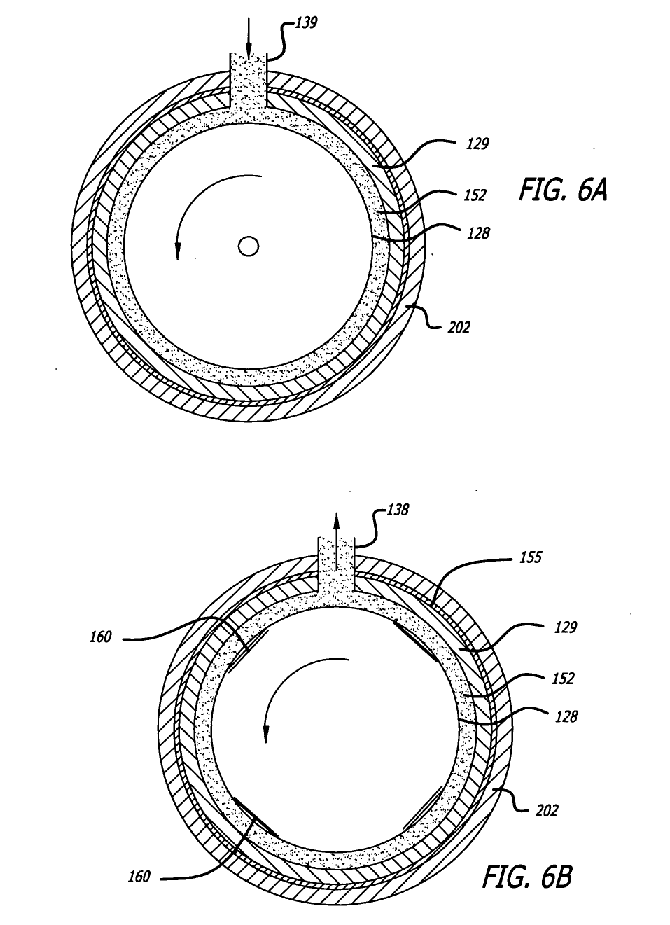

FIG. 6A illustrates a simplified illustrative cross section view in one embodiment of a portion of a shear treatment zone, that is, the annular gap/processing passage 152 reactor apparatus 100, in accordance with one aspect of the present disclosure. The cross section view shows the rotor 128 as the innermost circle in the diagram. The rotor 128 may or may not be hollow. The annular gap/processing passage 152 houses the passing cell culture that passes therethrough the reaction apparatus 100, via the primary inlet 139 or, as illustrated here, through an inlet that is substantially perpendicular to the annular gap/processing passage 152 and the axis of rotor 128 and stator 129. In another embodiment, the primary inlet 139 is parallel to the axis of the rotor 128, as illustratively shown in FIG. 2A, for example.

In one embodiment inlet 139 can also be configured to be “off-center” of the axis of rotor 128 and stator 129, but still parallel to the axis. In one embodiment, reactor apparatus 100 can be provided with a combination of inlets to the annular gap/processing passage 152, having one inlet “in-line” and/or parallel with the axis of rotor 128 and stator 129 and one inlet that is substantially perpendicular to the longitudinal axis of the rotor 128 and stator 129 and annular gap/processing passage 152.

As stated above, FIG. 6A is a simplified cross section of a view in one embodiment of a portion of shear treatment zone. FIG. 6A also depicts a portion of outer jacket 202 that surrounds the rotor 128 and stator 129 and can define at least a portion of a circumnavigating conduit for flowing of a temperature control fluid, such as a heat exchange fluid 155, that encircles and transfer energy, such as heat, either to or from the cell culture flowing through the annular gap/processing passage 152 via energy transfer through stator 129.

The annular gap/processing passage 152 provided between the outer surface of rotor 128 and the inner surface of the stator 129 can be larger than a few cell radii (i.e. large enough to prevent cell lysis). Of course, cell radii differ from cell-type to cell type, and thus the dimensions of annular gap/processing passage 152 are provided accordingly (relatively big cells being passed through a larger annular gap/processing passage 152 relative to an annular gap/processing passage 152 utilized for relatively smaller cells). For example, the diameters of mammalian cells are in the 10 μm to 50 μm range. As such and for example, five cell diameters would translate to 50 μm. Thus, a gap with a minimum of 50 μm can be large enough to prevent cell lysis. As discussed above, including in the discussion following Table 2, for any particular configuration or variable chosen the annular gap/processing passage 152 gap size should be chosen to maintain laminar conditions in the anticipated operating range.

FIG. 6B also illustrates a cross section view of another point of a shear treatment zone of the in the reactor apparatus 100 including process outlet 138. As discussed above and in one embodiment, a disclosed perfusion method combines features of conventional centrifugation and the filtration approaches. The reactor apparatus 100 achieves such combination by allowing the medium to pass out of the circulating system through at least one opening or a plurality of openings in rotor 128 that are covered with a membrane or other type of cell-retaining filter.

Since shear forces on cells in the culture as they pass through annular gap/processing passage 152 will move them outward and away from rotor 128 and toward the wall of stator 129, it is possible to remove media through an orifice in the rotor 128 (perfusion) without the orifice becoming plugged with cells. Cells are prevented from plugging the filter by the mild centrifugal force acting in the annular gap/processing passage 152. A membrane 160 would be placed over a provided opening, or plurality thereof, in the rotor 128, which can be circular, square, or elongated, or any useful shape. This will allow media and any desired material (e.g. proteins) produced by cells of the cell culture, to be continuously harvested and media to be continuously refreshed with newly added media, added, for example to the vessels of cell culture apparatus 40.

The controlled amount of stress exerted on the cells induces cell stasis and/or increased protein production. Thus in one embodiment, it is desired that the reactor apparatus 100 operate near the edge of the maximal tolerated stress range. The stress on the cells is a product of the shear force that cells experience in the reactor apparatus 100, as they pass through the annular gap/processing passage 152. The shear force experienced by the cells is uniform and universal. In the reactor apparatus 100 of the present disclosure, all cells spend an equivalent amount of time experiencing the same degree of shear force. In contrast, the shear force that a cell experiences in standard stirred tank bioreactor will depend on how close it comes to a mixing paddle, and how long it spends in a mixing eddy. Furthermore and as disclosed herein, all cells of a cell culture pass through the reactor apparatus 100, via the annular gap/processing passage 152, regularly and evenly. In contrast, prior art methods provide great variability in how often, or even whether, a given cell in a stirred tank comes close enough to the mixing paddle to experience significant shear force in a conventional system.

The profile, duration and uniform application the shear force provided by the reactor apparatus 100, and thus, the nature of cell stress experienced by cells in cell culture, cannot be reproduced by conventional bioreactor systems.

There is no practical way of placing all cells in a uniform force zone for a selected period of time using conventional reactor technologies. For example, in a conventional, stirred tank bioreactor, if higher shear force is produced using more energetic mixing, the time of exposure is reduced since the cells are more rapidly propelled away from the mixing zone. Furthermore, each cell will experience this force differently, if at all, since passing close to the mixing blades is a probabilistic event. In contrast, by utilizing the reactor apparatus 100 of present disclosure, any desired force profile can be uniformly and reproducibly applied to cells.

Further, temperature control of a cell culture, as it passes through an annular gap/processing passage, is precisely controlled via an energy/heat exchanger 200. Heat exchange fluid 155 surrounds the stator 129 in order to provide for accurate temperature control and maintenance of the cell culture as it passes through the annular gap/processing passage 152.

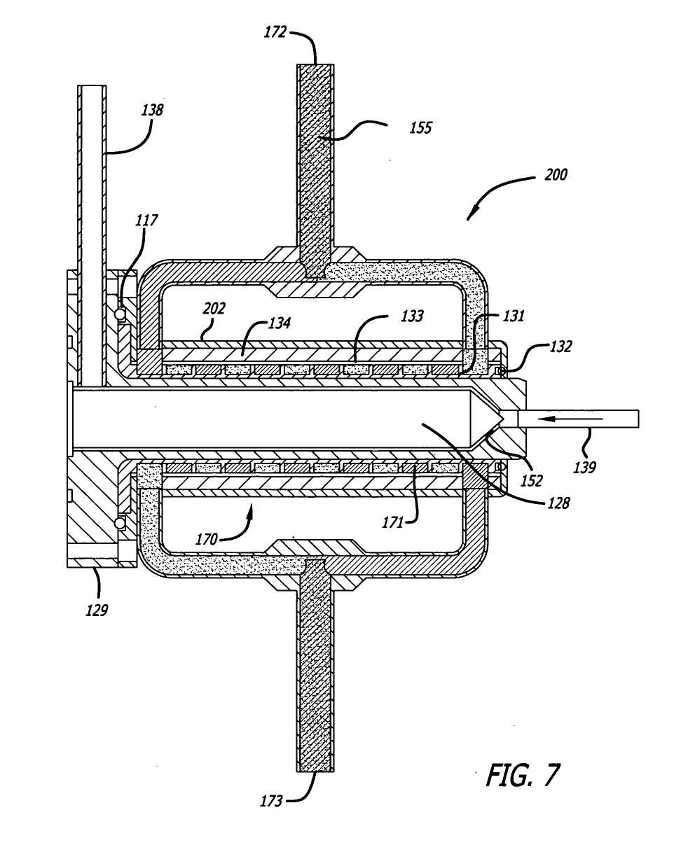

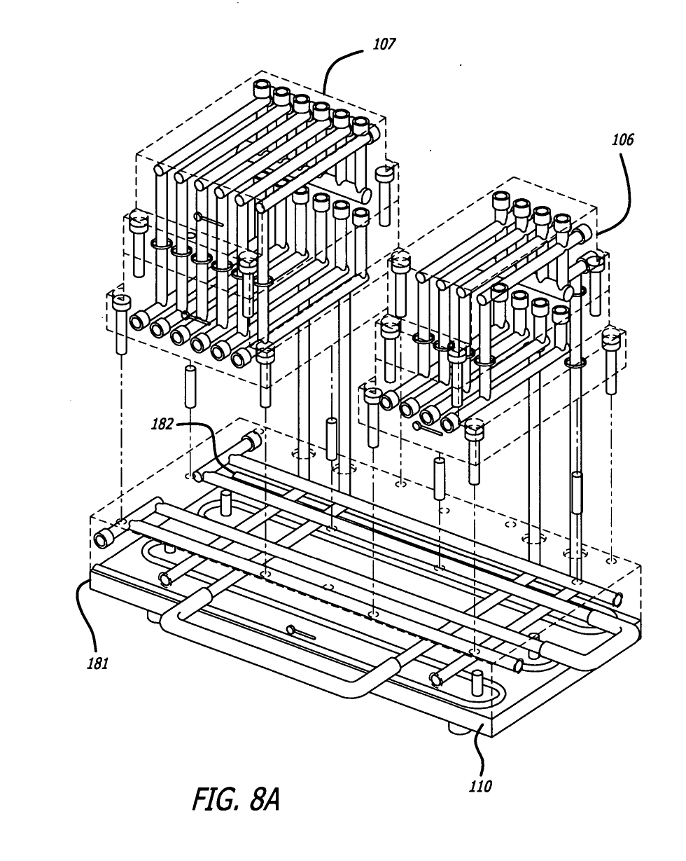

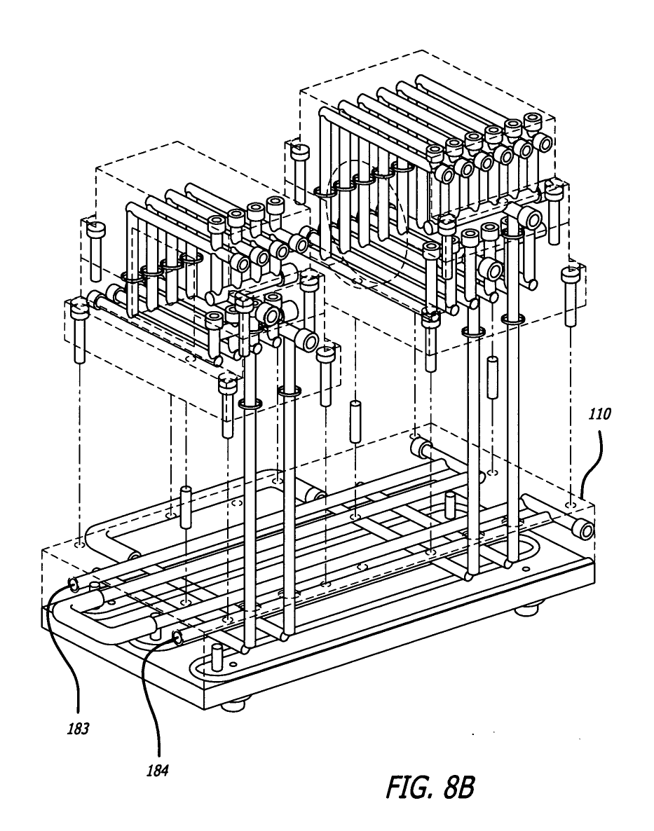

FIG. 7 illustrates a half section view of the heat exchanger system 200. The heat exchanger system 200 comprises a heat exchanger fluid inlet 172 and a heat exchanger fluid outlet 173. The heat exchange fluid 155 circulates in a spiral flow path around the stator 129 through the heat exchanger 170. Twin conduits 171 guide the fluid in a spiral path around the stator 129. The heat exchange fluid 155 allows for the control of the temperature of the cell culture in the annular gap/processing passage 152.

In one embodiment, and as seen in FIG. 7, outer jacket 202 surrounds a TEFLON® (comprised of polytetrafluoroethylene, or PTFE, or similar high temperature polymer such as PEEK®) insulator 134, which is adjacent to a sleeve 133, which can be made of a metal, such as stainless steel. In this embodiments, sleeve 133 forms a portion of the spiral conduit that defines a flow path for heat exchange fluid 155 and is adjacent to stator 129.

In one embodiment, the fluid is released to the twin conduits 171 on the same end such that the fluid in each grove circulate in the same spiral direction around the stator 129. In another embodiment, the twin conduits 171 receive the fluid on opposite ends, in such a way that the liquid in one twin conduit 171 flows in an opposite—counter current spiral direction as the liquid in the other twin conduit 171. That is, if viewed from one end the heat exchange fluid 155 in one conduit flows in a clock-wise direction and the fluid in the other conduit flows in a counter-clockwise direction. As shown in FIG. 7, heat exchange fluid 155 enters heat exchanger fluid inlet 172, the flow then splitting between left and right conduits that lead to respective conduits that circumnavigate the rotor 128 and stator 129, as illustrated by the hatched and stippled portions of heat exchange fluid 155 flow, indicated in FIG. 7. Fluid flow entering the left-hand portion of heat exchanger system 200, for example, proceeds around stator 129 in a spiral fashion and exits a right-hand portion of heat exchanger 200 (see and follow hatched 171 conduit), while fluid flow entering the right-hand portion of heat exchanger system 200, for example, proceeds around stator 129 in a spiral fashion (counter currently relative to hatched 171 conduit) and exits a left-hand portion of heat exchanger 200 (see and follow stippled 171 conduit), both flows (hatched and stippled 171 conduits) meeting up, mixing together and at exiting via heat exchanger fluid outlet 173 to a heater/temperature regulator (note shown). The conduits in this particular embodiment provide counter current flow of heat exchange fluid 155 around stator 129 and thus provides accurate control of the temperature of a cell culture media as it passes through annular gap/processing passage 152 such that it is maintained at a desired temperature.

For a cell culture, the temperature is preferably maintained to within 1° C., or better of a target temperature. Such accuracy and temperature maintenance is accomplished by passing temperature control fluids not only through the spiral conduits of heat exchanger 170 surrounding the stator 129, but also through various parts of body of the reactor apparatus 100. Such heat exchange fluids can be provided by separate conduits and temperature controls (not shown) as the temperature control requirements and parameters for a motor chiller block and bearing block typically differ from the heat exchanger 170, for example.

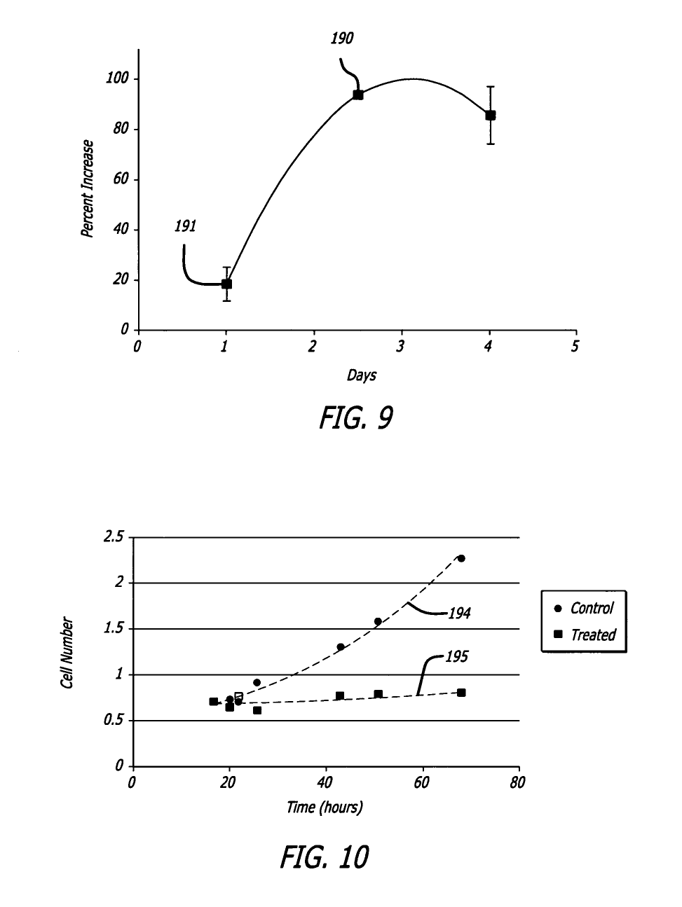

FIGS. 9A and 9B illustrate conduits defining an exemplary path of cooling/heating fluid through various portions of the body reactor apparatus 100. The cooling/heating fluid flows through the body of reactor apparatus 100 guided by a plurality of pipes, holes or passages drilled in a matrix pattern located in the motor chiller block 106, the bearing chiller block 107 and the base plates 110 portions of reactor apparatus 100.

In one embodiment, the cooling/heating fluid is the same fluid that is passed through the spiral conduits of heat exchanger 170, located about the stator 129. In another embodiment, the cooling/heating fluid is a different fluid than that passed through the heat exchanger 170 and around the stator 129. The cooling/heating fluid may be added to the motor chiller block 106 and the bearing chiller block though a fluid inlet 183 located in the base plate 110. The fluid inlet 183 may have an extension shown in FIG. 2A as cooling/heating fluid inlet 135. The cooling/heating fluid may be released though a fluid outlet 184 also located in the base plate. In one particular embodiment, cooling/heating fluid is transported/moved through the conduits and maintained at a desired temperature by least one appropriate pump and thermal regulator (not shown) that form part of a circulating system of cooling/heating fluid traveling from the at least one pump and thermal regulator (heater/cooler) to the motor chiller block 106 and the bearing chiller block of reactor apparatus 100 and back to the at least one pump and thermal regulator.

The temperature control fluids (e.g. heating/cooling fluid) can be a mixture of water and propylene glycol. In another embodiment, the heating/cooling fluid can be a light oil. In particular, embodiments, a hollow rotor 128 with heating fluid pumped therein can be utilized to exchange heat energy from the heating fluid, via walls of the rotor 128 and into the cell culture which is in contact with the outer surface of the rotor 128, which faces the annular gap/processing passage 152.

The conduits (e.g. pipes or passages) can be made of aluminum or similar material because of its desirable heat transfer qualities. In another embodiment, copper also can be used for components such as the motor bearings and heat exchangers. Copper has almost twice the coefficient of heat transfer of aluminum, thus assisting to the overall heat dissipation of the system. Other temperature control means, such as bolt-on heat exchanger tubes to control incoming or outgoing process chemicals, may also be used for some applications. Oil, alcohol or fluid such as, but not limited to, water can be used as a means of temperature control when pumped through a precision temperature control bath or baths designed to control temperature in the bath within or better than 0.1° C. Such baths are not shown and are also vendor purchased items such as a JULABO F33 or F series heating/cooling unit.

### EXPERIMENTS

Cells utilized in the cell culture experiments disclosed herein had been selected for optimal expression of a cloned protein of therapeutic interest. The parental cell line had also been selected for low aggregation characteristics, however, a few large clumps of cell were observed during the course of various experiments.

When mention is made to “treated cells,” it is to be taken that this is to mean that cells and/or medium containing cells, that have passed through the annular gap/processing passage 152 of the illustrative reactor apparatus 100 of the present disclosure.

It is contemplated that any cell line that can be maintained in a suspension cell culture will benefit from and can be utilized in accordance with the systems, methods and apparatus disclosed herein. Exemplary cells include, but are not limited to, mammalian cells, such as Chinese hamster ovary (CHO cells) cells, simian fibroblast CV-1 cells transformed by SV40 deficient in origin of replication region (COS cells), human cell lines (HEK 293, CEM), mouse, dog, stem cell lines as well as others. Exemplary cell lines also include insect cell lines such as Sf9, Sf21 and S2, for example. Culturing of fungal and yeast cells can also benefit from the present disclosure, for example, fermenter yeast, Chrysosporium lucknowense, and Pishia pastoris, as well as the culturing of bacterial cells, such as Escherichia coli cell lines. An increase of primary metabolite production in fermenter yeast has been attained.

Results provided herein indicate that cell proliferation can be maintained at control levels or attenuated, depending on the operating parameters of the reactor apparatus 100, particularly rate of culture flow and provided shear force. For example, cell proliferation rates can be maintained at or near control rates when the cells and the media are introduced at the distal port on the reactor apparatus 100.

The shear force exerted on cell culture, via passage through annular gap/processing passage 152 of the reactor apparatus 100, significantly affects cell proliferation. The symmetrical system (rotor 128 and stator 129 mounted concentrically) provides a uniform shear force around the rotor, while an asymmetrical system and mounting (eccentric alignment of rotor 128 and stator 129) has a higher shear force in the narrower gap than at a wider gap.

As such, if the reactor apparatus 100 is not a symmetrical system, the direction of culture flow affects both the nature and the degree of a shear force that results from rotation of rotor 128 in stator 129. Subsequent experiments were run under conditions where 1) cell proliferation/division was maintained at or near control levels and 2) conditions at which cell proliferation/division was attenuated.

Shear rate is influenced by multiple factors such as the gap size of annular gap/processing passage 152, the rotor 128 diameter, the fluid involved, revolutions per minute by the rotor 128, etc. as discussed above. For example, rotor speeds between 250 and 1600 rpm were utilized, with an optimal speed for the present experiments and conditions appearing to be 450 rpm for a 15.5 mm diameter rotor. Of course, optimal rotor speeds are culture specific and can vary depending upon cell count in the cell culture, the viscosity of culture media, type of media, the robustness of the particular cells in suspension (some cells being able to withstand a higher level of shear forces than others) etc. Optimal rotor speeds are easily determined for the particular set of conditions at hand. In particular embodiments contemplated useful rotor speeds vary in order to maintain laminar flow conditions, i.e. Taylor vortices are not induced.

In conditions under which division was maintained at or near control levels, a modest increase in protein expression in cultures that were treated (i.e. passed through the annular gap/processing passage 152 of reactor apparatus 100), compared to controls which are maintained in shaking flasks, was observed in as few as twenty-four hours.

FIG. 9 illustrates a diagram depicting increased protein production of treated cell cultures when compared to controls, in accordance with principles of the present disclosure. In particular, the diagram illustrates the percent increase in protein production over time. The cell type utilized during experiments and for which data is provided is a transfected Chinese hamster ovary (CHO) cell line (DG 44) which was grown in a serum free medium (IC CHO CT, Irvine Sciences) and expressed a human antibody protein; namely, a single-chained human antibody. Similar results were achieved with CHO cells expressing human tissue plasminogen activator (TPA). The parental CHO cells used in the experiments detailed herein had been adapted to suspension growth and selected for their ability to grow without forming large aggregates. The transfected cells were clonally selected for high expression of a protein encoded by a transfected sequence (transgene).

The increase in transgene protein production appeared to peak at around 60 hours but was still present at 96 hours. At its peak, the increase in protein production induced by treatment (passage of the cell culture through the annular gap/processing passage 152 of reactor apparatus 100) was nearly two-fold. As the error bars (SEM) indicate, this effect exhibited moderate variability. Quantization of protein expression in these experiments was performed by ELISA. The expressed protein was recognized by both monoclonal and polyclonal antibodies, and implies that the structure of the protein was not grossly affected by treatment of the cells in accordance with the present disclosure, that is, passage through the annular gap/processing passage 52 of reactor apparatus 100.

Under conditions in which cell division was attenuated, a different pattern emerges. These experiments with attenuated cell division maintained a cell culture temperature of thirty-seven degrees Celsius, a rotor speed of 450 rpm, and the cell culture experienced a residence time of four seconds in the annular gap/processing passage 152 (of about 4 inches) of the reactor apparatus 100. The rate of cycling of cell culture through reactor apparatus 100 was between about seven to fifteen times per hour.

Other rates of cycling are contemplated, depending, of course, on the particulars of the cell culture utilized. Rates of anywhere from about two to about fifty times per hour or more are contemplated. Any cycling rate wherein cells of the cell culture are passed through the annular gap/processing passage 152 of reactor apparatus 100 a plurality of times is useful. Cell culture temperature can be maintained at any desired useful temperature, in accordance with desired cell culture and conditions and the teachings provided herein. The total impact of the shear force that cells experience as they pass through the annular gap/processing passage 152 of reactor apparatus 100 is a combination of shear rate, rates of cycling and residence time within reactor apparatus 100.

FIG. 10 illustrates a diagram depicting increases in treated cell number under cell culturing conditions in accordance with the present disclosure, compared to control cell-culturing conditions. The diagram shows the number of cells in the treated culture (i.e. passing through the annular gap/processing passage 152 of reactor apparatus 100) increasing slowly (connecting line 195), compared to the exponential increase in cell number in the control culture, in shaker only (connecting line 194). The cell number in the diagram increases by millions of cells per milliliter.

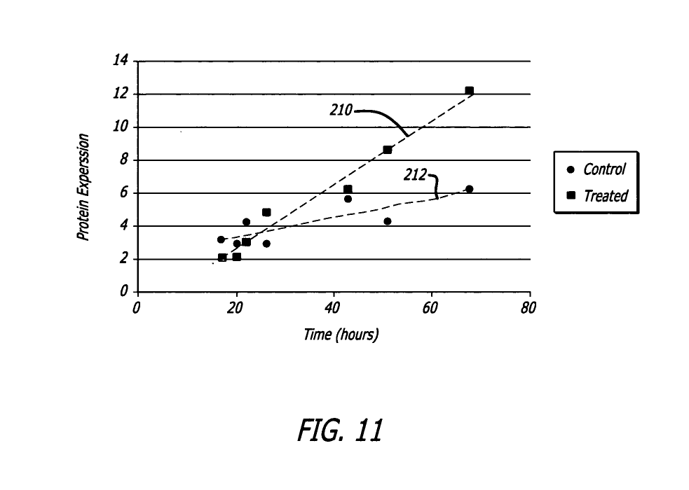

FIG. 11 illustrates a diagram exemplifying increased desired material or compound production by cells in a cell culture, here exemplified by protein production. For both cells proliferating at control/normal rates and cells proliferating at attenuated proliferation rates, the amount of protein secreted per cell increased linearly with time. Connecting line 212 shows control rates. The linear increase in both occurred as a result of the stability of the expressed protein in culture, the protein not being removed during the course of the experiment. However, the greater slope of the connecting line 210 in the treated culture indicated more protein production per cell per unit time than in the control culture depicted by connecting line 212. Interestingly, the control cells appear to show greater protein production at shorter intervals, which was surpassed by treated cells beginning at around forty hours. The diagram depicts a constant rate of protein production, seen as a linear increase in total protein expressed per cell.

While the above description contains many particulars, these should not be considered limitations on the scope of the disclosure, but rather a demonstration of embodiments thereof. The alloy, method for making and uses disclosed herein include any combination of the different species or embodiments disclosed.

For example, reactor apparatus 100 disclosed herein can be configured to easily connect and disconnect from existing cell culture systems, such as bioreactors. Because the reactor apparatus 100 uses a novel mechanism of enhancing protein production, it may be supplementary to other production-enhancing measures. In the experiments discussed above, the cells had already been optimized for protein production under standard culture conditions.

Similarly, the rotor 128, stator 129, and seal 116 can be removable as a single, sealed unit. Thus, after being autoclaved (or otherwise decontaminated utilizing standard techniques known in the art such as irradiation, etc.) they could be reinstalled without compromising their sterility.

Accordingly, it is not intended that the scope of the disclosure in any way be limited by specific embodiments. The various elements of the claims and claims themselves may be combined any combination, in accordance with the teachings of the present disclosure, which includes the claims.

1. A cell culturing system, comprising:
a cell culture including cell culture media and a plurality of cells;
a cell culture apparatus containing and maintaining said cell culture; and
a reactor apparatus connected to the cell culture apparatus, the reactor apparatus including a chamber wherein the chamber is an annular gap defined by a rotor and a stator wherein at least a portion of said cell culture is received and is submitted to a shear force in the absence of Taylor vortices.

2. A cell culturing system as defined in claim 1 wherein the reactor apparatus is connected to the cell culture apparatus by a pump for transporting said at least portion of said cell culture from said cell culture apparatus via a first conduit operably connected to said cell culture apparatus and to at least one inlet of said reactor apparatus.

3. A cell culturing system as defined in claim 1 wherein the chamber includes at least one cell-retaining filter to retain cells of said cell culture in said chamber while allowing for the passage of cell culture media.

4. A cell culturing system as defined in claim 2, further comprising:
an outer shell proximally disposed to an outer wall of said stator to define a temperature control passage configured for passage of a heat exchange fluid; and
a second conduit in operable communication between a processing outlet of said reactor apparatus and said cell culture apparatus.

5. The cell culturing system of claim 1, wherein said cell culture is a suspension cell culture and wherein said cells are selected from the group consisting of mammalian, plant, yeast, bacterial or fungal cells.

6. The cell culturing system of claim 5, where said cells include at least one transgene.

7. The culturing system of claim 1, wherein a temperature of said cell culture passing through said chamber is maintained to within about 0.1 to 1 degree centigrade of a desired cell culture temperature.

8. The culturing system of claim 2, wherein said rotor includes conduits that receive cell culture media removed from said annular gap via passage through the at least one cell-retaining filter in communication with said rotor.

9. The culturing system of claim 2, wherein at least one of said rotor or stator is made up of a material that is sterilizable.

10. The culturing system of claim 9, wherein said material is one of a high performance alloy, stainless steel, aluminum or aluminum alloy.

11. A method for culturing cells, comprising:
providing a cell culture including cells and cell culture media;
providing a cell culturing apparatus into which said cell culture is disposed;
providing a reactor apparatus in operable connection with said a cell culturing apparatus;
removing at least a portion of said cell culture from said cell culturing apparatus to said reactor apparatus;
passing said portion of said cell culture through a chamber provided by a rotor and a stator of said reactor apparatus, wherein either one or both of rotor and stator rotate relative to each other;
subjecting said portion of said cell culture to a shear force within said chamber, said shear force provided by rotation of either or both said rotor and stator and in the absence of Taylor vortices; and
returning said portion of said cell culture to said cell culturing apparatus.

12. A method for culturing cells as in claim 11, wherein said chamber is an annular processing gap.

13. The method of claim 11, further comprising the step of maintaining said portion of said cell culture, as said portion of said cell culture passes through the chamber, at a temperature that this substantially similar to cell culture temperature within said cell culturing apparatus from which said portion of said cell culture originates.

14. The method of claim 11, further comprising the step of collecting a cell culture product, produced by cells of said cell culture.

15. The method of claim 14, wherein said collecting step includes the step of removal of at least a portion of said cell culture media from said cell culture.

16. The method of claim 15, wherein said collecting step of said portion of said cell media includes a perfusing step, wherein additional cell culture media is added to said cell culture.

17. The method of claim 16, wherein said removed portion of said cell culture media from said cell culture is removed via passage through a cell-filtering membrane which provides transport of cell culture media and not cells therethrough.

18. The method of claim 16, wherein said cell-filtering membrane is disposed on said rotor or said stator or both.

19. The method of claim 16, further comprising the step of collecting a cell culture product, produced by said cells of said cell culture that is carried by said removed portion of cell culture media.

20. The method of claim 11, wherein said cells of said cell culture contain at least one transgene.

21. The method of claim 13 or 19, wherein said cell culture product is a protein.

22. The method of claim 1, wherein said cells are mammalian or plant or bacterial or fungal cells.

23. A cell culture product produced in accordance with the method of claim 11.

24. A reactor apparatus for cell culturing, comprising:
a stator having a generally cylindrical shape;
a rotor having a generally cylindrical shape, the rotor being located within the stator;
the inside surface of the stator and the outside surface of the rotor defining an annular gap having a gap width;
wherein the annular gap is adapted to receive a cell culture medium containing cells;
wherein the rotor is adapted to rotating within the stator, wherein rotation of the rotor imparts a shear force to the cells; and
wherein the gap width is configured so that as the rotor rotates Taylor vortices are not induced.

25. The reactor apparatus of claim 24 wherein the cell culture medium has a viscosity and the gap width is configured as a function of the cell culture viscosity and the rotation speed of the rotor.

26. The reactor apparatus of claim 24 wherein the cell culture medium has a viscosity and the gap width is configured as a function of the cell culture viscosity, the radius of the rotor and the rotation speed of the rotor.

27. The reactor apparatus of claim 24 wherein the cell culture medium has a viscosity and a fluid density, and the gap width is configured as a function of the cell culture viscosity, the rotation speed of the rotor and the fluid density of the cell culture.

28. A reactor apparatus for cell culturing, comprising:
a stator having a generally cylindrical shape;
a rotor having a generally cylindrical shape, the rotor being located within the stator;
the inside surface of the stator and the outside surface of the rotor defining an annular gap having a gap width;
wherein the annular gap is adapted to receive a cell culture medium containing cells;
wherein the cell culture medium has a viscosity and a fluid density;
wherein the rotor is adapted to rotating within the stator, wherein rotation of the rotor imparts a shear force to the cells; and
wherein the gap width, rotation speed of the rotor and radius of the rotor are chosen in view of the cell culture viscosity and fluid density so that as the rotor rotates Taylor vortices are not induced.

29. A method for culturing cells, comprising:
providing a cell culture including cells and a cell culture medium wherein the cell culture medium has a viscosity and a fluid density;
providing a reactor apparatus including a stator having a generally cylindrical shape and a rotor having a generally cylindrical shape, the rotor being located within the stator and the inside surface of the stator and the outside surface of the rotor defining an annular gap having a gap width wherein the annular gap is adapted to receive a cell culture medium containing cells;
rotating the rotor within the stator, whereby the rotation of the rotor imparts a shear force to the cells; and
providing a gap width, rotation speed of the rotor and radius of the rotor as a function of the cell culture viscosity and fluid density so that as the rotor rotates Taylor vortices are not induced.
